(19) United States
(12) Patent Application Publication
(10) Pub. No.: US 2023/0030410 A1
(43) Pub. Date: Feb. 2, 2023

(54) IMPLANT FOR TREATING BONES

(71) Applicant: Medartis Holding AG, Basel (CH)

(72) Inventor: Andreas MULLIS, Hemmiken (CH)

(21) Appl. No.: 17/784,199

(22) PCT Filed: Dec. 9, 2020
(86) PCT No.: PCT/EP2020/085153

§ 371 Date: Jun. 10, 2022

(30) Foreign Application Priority Data
Dec. 13, 2019 (EP) 19216226.1

(51) Int. Cl. A61B 17/80; A61F 2/28

(52) U.S. Cl. CPC A61B 17/8085; A61F 2/2875; A61B 2017/00946

(57) ABSTRACT

The invention relates to an implant (1) for the treatment of bone, in particular for covering defects or drill holes or for the reconstruction of bone defects or malformations. This comprises at least one frame structure (2) and at least one adaptation area (3). The edge of the implant (4) is thereby partially, but not continuously, formed by the frame structures (2), which are located outside the adaptation area (3).

The invention relates to an implant for the treatment of bone with the features of the generic term of the independent claim. It is known that bone fractures, especially of the human skull, can be treated with implants. The aim of the treatment and implantation of an implant is generally to restore and maintain the anatomically correct shape and position of the bones to be treated and their surroundings. This promotes the healing process and preserves the functionality of the affected body part after healing has taken place.

In the prior art, various implants are known that are adapted to specific parts of the body.

EP 2 030 596 discloses an implant for the treatment of fractures of the orbit. These include a planar fitting area and a frame structure.

U.S. Pat. No. 5,139,497 discloses an orbital floor implant having a frame structure and a grid.

US 2007/0238069 discloses cuttable and deformable meshes for the treatment of facial fractures.

However, known implants have various disadvantages. For example, certain implants are equipped with frame structures that are adapted to specific bone areas. However, this sometimes makes implant placement difficult, especially since the exact anatomy of people naturally varies. At the same time, however, a certain degree of adaptation to the anatomy is desirable.

It is therefore the task of the present invention to avoid the disadvantages of the prior art, in particular to provide an implant which on the one hand is adapted to the anatomy of the body part to be treated but at the same time allows certain adaptations.

According to the invention, these and other tasks are solved with an implant according to the independent claim.

The implant according to the invention is particularly suitable for covering defects or drill holes or for reconstructing bone defects or malformations. It comprises at least one frame structure and a sheetlike adaptation area. The frame structure is arranged outside the adaptation area. It partially forms the edge of the implant. The frame structure does not, however, completely define the outer edge. This means that at least one area of the outer edge is not limited by the frame structure. This allows the frame structure of the implant to be adapted to a desired anatomy. At the same time, the adaptation area not limited by the frame structure allows cutting or deformation and thus further adaptation to the anatomy.

A frame structure is to be understood as a region of the implant, in particular an edge region, which is designed by means of di-mensioning and/or by material selection and/or shaping in such a way that a minimum force required for plastic deformation of the edge region, in particular preferably the edge region and the adaptation area together, is higher than a minimum force required for plastic deformation of the adaptation area. This property is referred to here and in the following as bendability. A smaller bendability of a part therefore means that a larger force must be applied to plastically deform it. A greater bendability of a part means that a smaller force is sufficient to deform it plastically. Accordingly, the frame structure may be less bendable compared to the adaptation area. Additionally or alternatively, a frame structure can also be a region of the implant, in particular an edge region, which interrupts the periodicity of the lattice.

The implant according to the invention is particularly suitable for the treatment of the human frontal sinus and is shaped and dimensioned accordingly. However, it can of course also be used for other parts of the body if the size and shape are suitable. Particularly preferably, the implant is mirror-symmetrical along a mirror plane.

Preferably, the implant comprises at least two frame structures. These are both arranged outside the adaptation area and thus partially form the edge of the implant. The two frame structures do not continuously delimit the outer edge of the implant. As a result, at least two areas of the outer edge are not bounded by the frame structure. In particular, this allows adaptation to bones that have two similar and/or symmetrical regions, with the intermediate region varying from person to person. For example, the two frame structures may be adapted to the areas around the eyes and have an adaptation area to adapt to the nasal bone.

Preferably, the adaptation area is continuous and is free of frame structures on the inside.

Preferably, the frame structure is dimensioned and arranged such that at least half of the outer edge is not bounded by the frame structure. In the present case, half of the outer edge means half of the length of the outermost edge of the implant.

Preferably, the adaptation area comprises a grid structure. The grid structure is dimensioned in such a way that it can be deformed by hand or with hand tools. In particular, the adaptation area can be designed so that its bendability is adapted appropriately.

In particular, to this end, the adaptation area may be made of or comprise a material having an strain at failure of at least 5%, preferably at least 10%, more preferably at least 15%.

Likewise, the adaptation area can be at least partially dimensioned so that at most a force of 300 N, preferably at most 150 N, particularly preferably at most 50 N, is required to achieve plastic deformation of the adaptation area, in particular a force acting vertically on the adaptation area. For example, the adaptation area can have wire- or rod-shaped elements with a area moment of inertia or material properties to achieve such deformabilities. Depending on the material properties, the dimensions or area moments of inertia of the rod-shaped elements can be different in order to achieve the same deflection for the same force. Similarly, it is conceivable to make the thickness of the adaptation area such that a maximum force, in particular a maximum force listed above, is sufficient to achieve plastic deformation. In particular, the adaptation area can be at most 2 mm, preferably at most 1 mm, especially preferably at most 0.6 mm thick. Particularly preferably, the adaptation area has a thickness of 0.25 mm, 0.4 mm or 0.6 mm. Preferably, the implant comprises a biocompatible material, in particular from the group of implant steel and/or other metals, ceramics and plastics. Particularly preferably, the implant comprises titanium or a titanium alloy.

Preferably, the adaptation area has connection areas that are dimensioned so that they can be cut through using hand tools. This allows a surgeon to easily cut the adaptation area to fit the patient's anatomy.

In particular, the connection areas can be designed as described above in relation to the adaptation area.

The adaptation area preferably comprises a structure of holes whose circumferential areas are connected to each other via convection areas. In particular, the connection areas can be designed as ribs that connect the peripheral areas of the holes at regular intervals along their circumference.

Preferably, the implant has a side length in a range of 10-200 mm. Alternatively, the side length may be in a range of 30-70 mm, or 25-50 mm. In particular, the implant may have an approximately square shape with rounded corner regions. Particularly preferably, the frame structure is arranged in two adjacent rounded corner regions.

Preferably, at least one frame structure is dimensioned and positioned such that it can be attached, in particular screwed, to the margo supraorbitalis.

To this end, the frame structure may have at least one, preferably two, regions that correspond substantially in shape, curvature and/or size to the margo supraorbitalis. For example, the frame structure may have a radius in this region that has a deviation of at most +/−30%, preferably at most +/−20%, particularly preferably at most +/−10% to the radius of the orbit. It is also possible for this region of the frame structure to have a radius that substantially corresponds to the radius of the orbit (i.e. has an approximately equal radius). Particularly preferably, the radius of this region is substantially the same as the radius of the orbit. Further, these regions may have a length that is at most equal to the diameter of the human orbit. For example, the length may be at most 80 mm, preferably at most 60 mm, particularly preferably at most 50 mm.

Preferably, the frame structure has an arc shape. In particular, the arc shape may have a radius of curvature of 10-200 mm, more preferably 10-80 mm. The radius of curvature may vary along regions of the frame structure. For example, the radius of curvature may increase continuously along an edge structure so that the frame structure forms the shape of a clothoid.

Particularly preferably, the implant has an intermediate region. This is arranged between the two frame structures adapted to the margo orbitalis and can have a greater bendability than the two frame structures.

Likewise, it is conceivable that the intermediate region thereby has in particular a length which essentially corresponds to the inner distance between two eye sockets of the human skull and/or the width of a human nose and/or the typical anatomical distance between the human lacrimal glands. Preferably, the connecting piece has a length of 0.5 to 4 cm, particularly preferably of 1 to 2 cm.

Preferably, the implant has at least one attachment tab. Alternatively, the implant can also have at least two attachment tabs. In particular, the attachment tab may be located at the edge of the implant or on a frame structure. Preferably, the attachment tab also has screw holes. This allows the implant to be fixed in a particularly advantageous manner, for example to be screwed to a bone. Preferably, the fixing tab extends away from the edge of the implant in the same plane as the implant, in particular at an azimuth angle between 70° and 95° with respect to the edge. In particular, if the edge of the implant is not straight at the point where a fastening tab is located, the azimuth angle specification should be understood as the angle between the fastening tab and a tangent to the edge of the implant at the point where the fastening tab is located. Particularly preferably, two attachment tabs extend away from the implant such that there is an acute angle between the attachment tabs that opens away from the implant.

Additionally or alternatively, one or more attachment tabs may also be arranged so that they do not lie in the same plane as the implant, as the edge of the implant, or as the adaptation area of the implant and therefore lie at an elevation angle to said plane. In particular, the attachment tab may be angled away from the plane in which at least the edge of the implant and/or a frame structure lies.

It is understood that implants are also conceivable which are not flat but have a free form. In particular, the implant can have local bends between planar elements, and/or have a shape of a rotational or translational surface. In this case, the aforementioned plane refers to a tangent surface to the surface of the implant at the location where the tab is attached. In particular, for tabs that are not in the same plane as the tangent surface, an elevation angle can therefore be measured between the tab and its projection on the tangent surface. An azimuth angle can be measured in the tangent plane between the tab and a tangent to the edge of the implant.

In particular, the at least one attachment tab may be integrally connected to the frame structure. The implant can also have at least two attachment tabs that are integrally connected to the frame structure. If the implant also has at least two frame structures, the at least two attachment tabs are preferably integrally connected to one frame structure each. However, it is also possible for more than one attachment tab to be integrally connected to the same frame structure.

Alternatively, however, it is also possible to arrange one or more attachment tabs at the edge of the implant without connecting them to the frame structure. In particular, one or two attachment tabs can be arranged between two frame structures. This is particularly advantageous if at least one frame structure is dimensioned and positioned in such a way that it can be attacked, in particular fastened with screws, to the margo supra-orbitalis.

Preferably, the at least one attachment tab is adapted for attachment to the nasal bone. The adaptation can be achieved in particular by the dimension, shape, and positioning on the implant. If the implant has at least two attachment tabs, preferably at least two attachment tabs are also adapted in the same way for attachment to the nasal bone.

In particular, the attachment tabs may have a length substantially equal to the length of the human nasal bone. For example, the attachment tabs can have a length of at most 3 cm, preferably at most 2 cm, particularly preferably at most 1.5 cm. Two attachment tabs may be arranged relative to each other such that their smallest distance corresponds to the width of the nasal bone. For example, the distance can be about 5-30 mm. Preferably, the attachment tabs are not arranged parallel to each other in this case. However, it is possible for the attachment tabs to be arranged in a plane at an angle greater than or less than 0°. Attachment tabs that are interlaced about their longitudinal axis are also conceivable.

If an implant has one or two areas adapted for attachment to the margo supraorbitalis, the attachment tabs can in particular be arranged at these areas. Preferably, one attachment tab is arranged on each of the areas adapted for attachment to the margo supraorbitalis. Particularly preferably, the attachment tabs arranged at the regions adapted for attachment to the margo supra-orbitalis, face each other. In this case, the implant may in particular be mirror symmetrical along a plane and/or axis substantially between the two attachment tabs and/or regions adapted for attachment to the margo supraorbitalis. However, non-mirror symmetrical designs are also conceivable.

Preferably, the adaptation area is formed in integrally.

Preferably, the entire implant is formed integrally.

Preferably, the implant is adapted to cover defects or burr holes or to reconstruct bone defects or malformations on the sinus.

Preferably, the adaptation area is plastically deformable. In particular, the adaptation area can be dimensioned or adapted in the choice of material so that plastic deformation is possible.

The invention is explained in more detail below with reference to the figures and embodiments, showing:

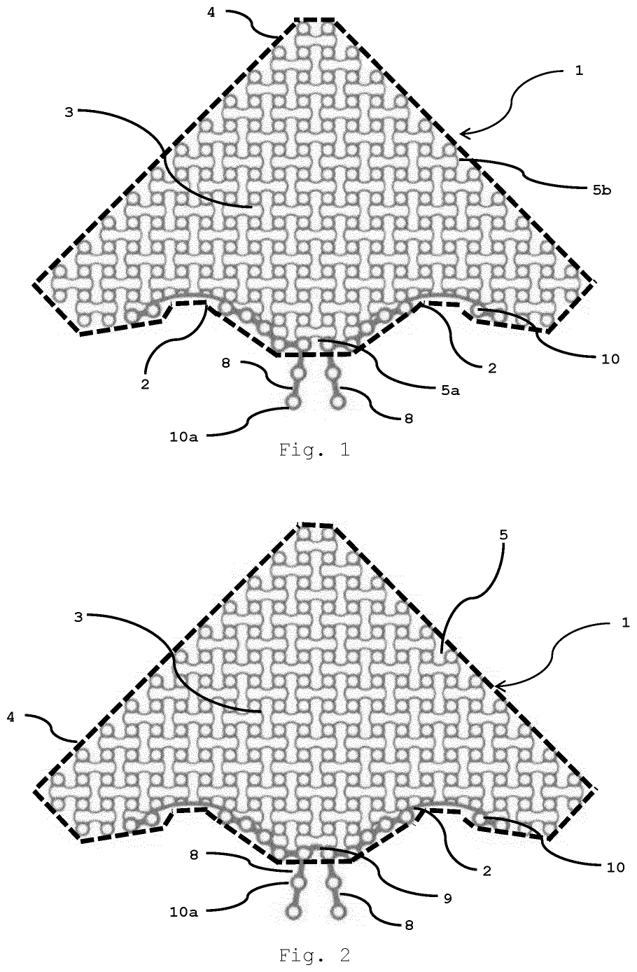

FIG. 1: an embodiment of an implant according to the invention,

FIG. 2: an alternative embodiment of an implant according to the invention,

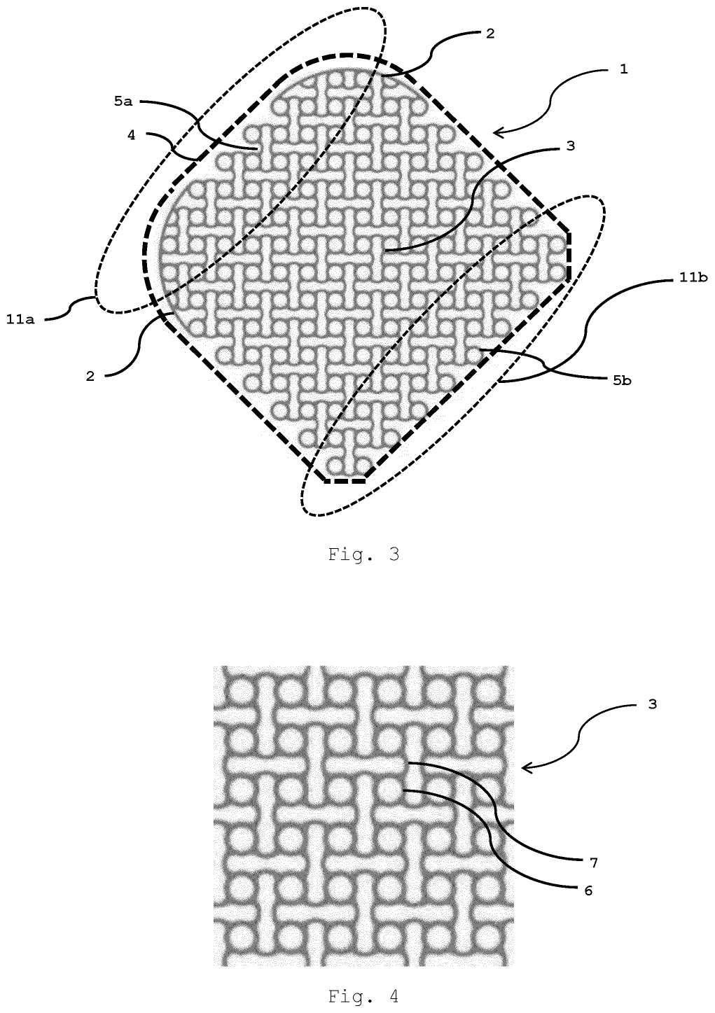

FIG. 3: a further alternative embodiment of an implant according to the invention,

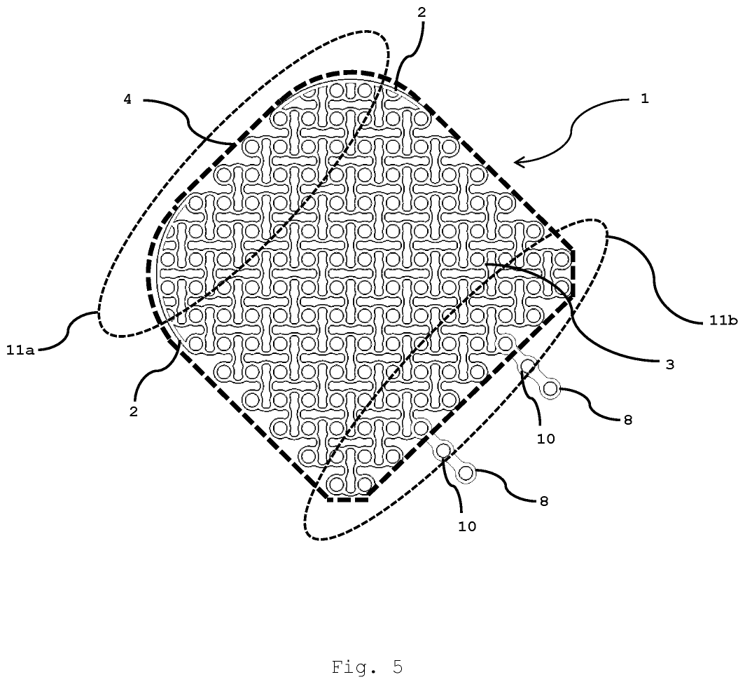

FIG. 4: an enlarged representation of a fitting area,

FIG. 5: an alternative embodiment of an implant according to the invention.

FIG. 1 shows an embodiment of an implant 1 according to the invention, which is adapted in shape and size to be implanted in the region of the human frontal sinus. The implant comprises two frame structures 2 and an adaptation area 3. The outer edge of the implant is symbolized by a dashed line 4 and is to be understood here and in general as the outermost boundary of the entire implant before any cutting. The two frame structures 2 partially form the outer edge 4 of the implant. In particular, the frame structures 2 here have an arcuate shape and are adapted in shape and dimension to the human margo supraorbitalis. Further, they comprise screw holes 10 suitable for receiving screws so that the implant 1 can be screwed to a bone. The implant further comprises two attachment tabs 8, each of which is integrally connected to one of the frame structures 2. In the present case, the attachment tabs 8 are connected at the facing ends of the frame structures 2 between the two frame structures and extend in the same plane as the implant at an angle of approximately 85° away from the implant. Thus, the attachment tabs 8 correspond to the position of the nasal bone when the frame structures are attached to the margo supraorbitalis. Further, the attachment tabs include screw holes 10a suitable for screwing the attachment tabs to the nasal bone. Between the two frame structures 2 and attachment tabs is an intermediate region 5a, which has no frame structure. This corresponds in its design to the remaining edge region 5b of the implant and is designed in particular in such a way that it has greater bendability than the frame structure. Thus, the force required to cause plastic deformation of the intermediate region 5a is relatively small and can be applied by hand or hand tools. In the present case, the implant comprises only frame structures adapted to the region of the margo supraorbitalis, which can be attached to the nasal bone via attachment tabs. The remainder 5b of the outer edge 4 is not bounded by frame structures and can therefore be trimmed by the surgeon. This allows, for example, the implant to be adapted to a smaller area of a patient's anatomy. In addition, the adaptation area 3 is plastically deformable, so that further adaptation possibilities exist.

FIG. 2 shows an alternative embodiment of an implant 1 according to the invention. The embodiment shown here comprises only one frame structure 2. The frame structure partially delimits the outer edge 4, so that a part 5 of the outer edge is formed without a frame structure. The sheetlike adaptation area 3 has essentially the same design as that shown in FIG. 1, and is therefore plastically deformable and has a bendability that permits plastic deformation by hand or with hand tools. The frame structure 2 is adapted here to correspond in shape and dimensions to the Margo Supraorbitalis. The implant has two attachment tabs 8, both of which are integrally connected to the frame structure 2. The attachment tabs are dimensioned and located so that they can be attached to the nasal bone. In particular, the screw holes 10a, which can be used for screwing to the nasal bone, serve this purpose. The frame structure also includes screw holes 10 which serve the same purpose. Here, the frame structure 2 is continuous between the two attachment tabs 8. Therefore, the intermediate area 9 between the attachment tabs is part of the frame structure 2. This is particularly advantageous if the treatment requires support and stabilization in this area and/or the shape and dimensions of the implant are so precisely adapted to the anatomy that further adaptation is unnecessary. Irrespective of this, however, the adaptation area 3 can of course be plastically deformed and/or cut to accommodate a particular anatomy.

FIG. 3 shows an alternative embodiment of an implant 1 according to the invention, which has two frame structures 2 that partially form the outer edge 4 of the implant. As a result, the implant comprises two further areas 5a, 5b of the outer edge 4 which are not bounded by frame structures. The sheetlike adaptation area 3 is also designed here so that it can be plastically deformed and cut to size. In particular, it is dimensioned and designed, for example by material selection and/or shape, such that plastic deformation and/or cutting can be performed by hand or with hand tools. Suitable hand tools include, in particular, commercially available pliers and cutting instruments. The implant has an approximately square shape with rounded corner regions with a radius, where the frame structures are also arranged. As a result, the present implant comprises a region 11a which has no sharp points or fraying, in particular due to the frame structure. A second region 11b, which in the present example is opposite region 11a, is particularly suitable for being cut to a smaller size by an operator because of the lack of frame structures. Preferably, the cutting is performed on the side facing away from the frame structures. However, it is of course also possible to cut the adaptation area to any shape, in particular also between the two frame structures. This results in a smaller implant with only one frame structure. The flexibility of the cutting makes this implant particularly suitable for use where precise adaptation of the implant prior to surgery is not possible or is difficult and flexibility in use is therefore important. For example, this is the case in the treatment of bones, which typically have a large variation in size and shape between individuals.

Alternatively, it would of course be conceivable to also cut the implant within the frame structure. The frame structure therefore does not have to be designed in such a way that it cannot be cut to size. In general, however, it is advantageous to arrange the frame structure in such a way that it does not have to be cut to size in the most common applications. In this way, the frame structure forms an area without sharp edges.

FIG. 4 shows a detailed representation of the sheetlike adaptation area 3 and the geometry of the lattice. The present grid comprises holes 6 and connection areas 7 designed as ribs. The connection areas connect circumferential areas of the holes 6. In the embodiment shown here, each circumferential area of a hole 6 is connected to four connection areas 7. These are evenly distributed along the circumference of the holes, i.e. approximately at 90° intervals. The connection areas are further dimensioned so that they can be cut through with hand tools. Here, the holes have an outer diameter of 3.1 mm. However, it would also be conceivable to form holes with a different outer diameter, in particular an outer diameter in the range from 2 to 5 mm, preferably in a range from 3.0 to 3.22 mm. The wire-like elements forming the ribs as well as the circumferential areas of the holes have a width of 0.6 mm, but could alternatively have a different width in the range 0.1 to 3.0 mm, preferably 0.5 to 0.7 mm. The grid area is approximately 0.5 mm thick, but could also have a different thickness in the range of 0.1 to 2.0 mm, preferably 0.3 to 0.6 mm. Particularly advantageous is the design of the grid from a metal or a (resorbable) plastic. Accordingly, the variant shown here is made of titanium or a titanium alloy. This design allows the sheetlike adaptation area to be cut to a desired size. However, other dimensions and/or materials can of course be used. The geometry of the grid forming the sheetlike adaptation area 3 shown here is particularly advantageous for use in an implant due to the properties described here. However, it is of course also possible to use any other known lattice structure.

FIG. 5 shows an alternative embodiment of an implant according to the invention. This corresponds essentially to the embodiment shown in FIG. 3, but further comprises two attachment tabs 8. These are similar to the attachment tabs used, for example, in the embodiment shown in FIG. 1. However, in the embodiment shown here, the attachment tabs 8 are not connected to a frame structure. Instead, both attachment tabs 8 are directly connected to the edge region 5b of the implant 1 and are therefore located in a region with greater bendability of the implant. This allows the implant 1 to be used particularly advantageously in complicated anatomies because the attachment tabs 8 can be fixed with maximum flexibility. For this purpose, the attachment tabs 8 also have screw holes 10. It would of course be possible to combine the arrangement of the attachment tabs 8 shown in FIGS. 1 and 5. Thus, the variant shown here could also additionally or alternatively comprise further attachment tabs 8 in the area of the frame structures. Likewise, the embodiment shown in FIG. 1 could be provided with further attachment tabs that are not connected to the frame structure.

1.-18. (canceled)

19. An implant for the treatment of bone, comprising:
at least one frame structure; and
at least one adaptation area;
wherein the at least one frame structure is arranged outside the adaptation area and partially forms the edge of the implant, wherein the at least one frame structure does not continuously delimit the outer edge, so that at least one region of the outer edge is not delimited by the frame structure.

20. The implant according to claim 19, wherein the implant comprises at least two frame structures, wherein both frame structures are each arranged outside the adaptation area and partially form the edge of the implant, and the at least two frame structures do not continuously delimit the outer edge, so that at least two areas of the outer edge are not delimited by the frame structure.

21. The implant according to claim 19, wherein the adaptation area is continuous and is free of frame structures on the inside.

22. The implant according to claim 19, wherein the frame structures are dimensioned and arranged such that at least half of the outer edge is continuously not delimited by the frame structures.

23. The implant according to claim 19, wherein the adaptation area comprises a lattice structure dimensioned, such that it can be deformed by hand or with hand tools.

24. The implant according to claim 19, wherein the implant comprises a biocompatible material.

25. The implant according to claim 19, wherein the adaptation area comprises connection areas dimensioned to be cut by means of hand tools.

26. The implant according to claim 19, wherein the implant has at least one side length in a range of 10-200 mm.

27. The implant according to claim 19, wherein at least one frame structure is dimensioned and positioned to be attached.

28. The implant according to claim 27, wherein the at least one frame structure has an arc shape.

29. The implant according to claim 27, wherein the implant has, between the two frame structures adapted to the margo supraorbitalis, an intermediate region whose bendability is greater than that of the frame structures.

30. The implant according to claim 19, wherein the implant comprises at least one attachment tab.

31. The implant according to claim 12, wherein the at least one attachment tab is integrally connected to a frame structure.

32. The implant according to one of claims 30, wherein the at least one attachment tab is adapted for attachment to the nasal bone.

33. The implant according to claim 19, wherein the implant is adapted to cover defects or drill holes or to reconstruct bone defects or malformations at the sinus.

34. The implant according to claim 19, wherein the adaptation area is formed in integrally.

35. The implant according to claim 19, wherein the entire implant is formed integrally.

36. The implant according to any claim 19, wherein the adaptation area is plastically deformable.
